(19) United States
(12) Patent Application Publication
(10) Pub. No.: US 2009/0208428 A1
(43) Pub. Date: Aug. 20, 2009

(54) Bioactive Glass

(75) Inventors: Robert Graham Hill, Berkshire (GB); Molly Morag Stevens, London (GB)

Correspondence Address: Pepper Hamilton LLP, 400 Berwyn Park, 899 Cassatt Road, Berwyn, PA, 19312-1183, US

(73) Assignee: IMPERIAL INNOVATIONS LIMITED, London (GB)

(21) Appl. No.: 12/304,790

(22) PCT Filed: Jun. 15, 2007
(86) PCT No.: PCT/GB07/02262

§ 371 Date: Apr. 2, 2009

(30) Foreign Application Priority Data
Jun. 16, 2006 (GB) 0612028.1

(51) Int. Cl. A61K 8/21; C03C 3/112; C03C 3/097; C03C 3/078; A61K 8/27; A61K 8/25; A61K 8/24; A61Q 11/00

(52) U.S. Cl. 424/52; 501/57; 501/63; 501/72; 424/53

(57) ABSTRACT

The present invention relates to a bioactive glass comprising strontium and silicon dioxide, processes for the production of the bioactive glass and the use of the bioactive glass in medicine.

The present invention relates to a bioactive glass comprising strontium, processes for the production of the bioactive glass and the use of the bioactive glass in medicine.

A biologically active (or bioactive) material is one which, when implanted into living tissue, induces formation of an interfacial bond between the material and the surrounding tissue. More specifically, bioactive glasses are a group of surface-reactive glass-ceramics designed to induce biological activity that results in the formation of a strong bond between the bioactive glass and living tissue such as bone. The bioactivity of silicate glasses was first observed in soda-calcia-phospho-silica glasses in 1969, resulting in the development of a bioactive glass comprising calcium salts, phosphorous, sodium salts and silicon. These glasses comprised SiO₂ (40-52%), CaO (10-50%), Na₂O (10-35%), P₂O₅ (2-8%), CaF₂ (0-25%) and B₂O₃ (0-10%). A particular example of a SiO₂—P₂O₅—CaO—Na₂O bioactive glass is manufactured as Bioactive Glass®.

The bioactivity of bioactive glass is the result of a series of complex physiochemical reactions on the surface of the glass under physiological conditions. When exposed to body fluid cation exchange occurs, wherein interstitial Na⁺ and Ca²⁺ from the glass are replaced by protons from solution, forming surface silanol groups and non-stoichiometric hydrogen-bonded complexes. The interfacial pH becomes more alkaline and the concentration of surface silanol groups increases, resulting in the condensation polymerisation of silanol species into a silica-rich surface layer. The alkaline pH at the glass-solution interface favours the precipitation and crystallisation of a carbonated hydroxyapatite (HCA) phase. This is aided by the release of the Ca²⁺ and PO₄³⁻ ions into solution during the network dissolution process which takes place on the silica surface. The HCA crystallites nucleate and bond to interfacial metabolites such as mucopolysaccharides, collagen and glycoproteins. Incorporation of organic biological constituents within the growing HCA and SiO₂ layer stimulate bonding to living tissues. The ionic products of bioactive glass dissolution have been shown to stimulate osteoblast growth and differentiation by upregulation of genes with known roles in processes related to osteoblast metabolism and bone homeostasis, such as those genes encoding products that induce osteoblast proliferation and promote cell-matrix attachment.

The rate of development of the hydroxycarbonated apatite (HCA) layer on the surface of the glass provides an in vitro index of bioactivity. The use of this index is based on studies indicating that a minimum rate of hydroxyapatite formation is necessary to achieve bonding with hard tissues. (See, for example, Hench, Bioactive Ceramics, in Bioceramics: Material Characteristics Versus In Vivo Behavior (P. Ducheyne & J. E. Lemons, Eds., 1988), pages 54-71). Bioactivity can be effectively examined by using non-biological solutions that mimic the fluid compositions found in relevant implantation sites within the body. Investigations have been performed using a variety of these solutions including Simulated Body Fluid (SBF), as described in Kokubo T, J. Biomed. Mater. Res. 1990; 24; 721-735, and Tris-buffered solution. Tris-buffer is a simple organic buffer solution while SBF is a buffered solution with ion concentrations nearly equal to those of human body plasma. Deposition of an HCA layer on a glass exposed to SBF is a recognised test of bioactivity. When the glass particles are exposed to SBF, the rate of development of the HCA layer may be followed by the use of Fourier Transform Infra Red Spectroscopy, Inductively Coupled Plasma Emission Spectroscopy (ICP), Raman Spectroscopy or X-Ray Powder Diffraction (See, for example, Warren, Clark & Hench, Quality Assurance of Bioactive glass.sup.(R) Powders, 23 J. Biomed. Mat. Res.—App. Biomat. 201 (1989)).

The chemical nature of HCA lends itself to substitution resulting in, for example, the substitution of the hydroxyl groups with carbonate or halides such as fluoride and chloride. The HCA layer that forms is structurally and chemically equivalent to the mineral phase of bone and allows the creation of an interfacial bond between the surface of the bioactive glass and living tissue. Hydroxycarbonated apatite is bioactive, and will support bone ingrowth and osseointegration.

Bioactive glasses have therefore found medical applications in the preparation of synthetic bone graft materials for general orthopedic, craniofacial, maxillofacial and periodontal repair, and bone tissue engineering scaffolds. The bioactive glass can interact with living tissue including hard tissue such as bone, and soft connective tissue.

Bioactive glasses have been produced using both conventional glass production techniques, such as the melt quench method and, more recently, sol gel techniques as described in, U.S. Pat. No. 5,074,916 and U.S. Pat. No. 6,482,444, both of which discuss the production of bioactive glasses using the sol gel technique.

Since the development of Bioactive Glass®, there have been many variations on the original composition. Many bioactive silica glasses are based on a formula called ‘45S5’, signifying 45 wt % silicon dioxide (SiO₂), and a 5:1 molar ratio of calcium (Ca) to phosphorus (P). However, variation in the ratio of these components, and inclusion of other components such as boron oxide (B₂O₃) and calcium fluoride (CaF₂), has allowed modification of the properties of the bioactive glass, including the rate of dissolution and the level of bioactivity.

Currently available bioactive glass compositions have a number of limitations. Most bioactive glass compositions contain sodium oxide (Na₂O) and may also contain potassium oxide (K₂O). The incorporation of these compounds into the bioactive glass is advantageous for the production of the glass, as they reduce the melting temperature of the bioactive glass. This reduction in melting temperature allows production of the bioactive glass at lower energy levels and reduces damage to the production equipment.

However, the presence of the alkali metals, sodium and potassium, at high concentrations in the bioactive glasses can reduce the usefulness of the bioactive glass in vivo. In particular, bioactive glass composites based on bioactive glasses having a high alkali metal content are susceptible to water uptake by osmosis resulting in swelling and cracking of the polymer matrix and may, in the case of degradable polymer composites, exhibit increased levels of degradation. Such bioactive glasses may be unsuitable for use as coatings for metal prosthetics due to the increased thermal expansion co-efficient of the bioactive glass as a result of the presence of the alkali metals. Furthermore, high levels of alkali metals make the bioactive glasses unsuitable for use in the manufacture of bioactive porous scaffolds and bioactive glass coatings, as the presence of high levels of alkali metals reduces the difference between the Glass Transition Temperature (T_{g}) and the onset temperature for crystallisation of the bioactive glass, leading to crystallisation during sintering of the glass and a general subsequent reduction in bioactivity.

Alternative bioactive glasses having lower levels of alkali metals are known in the art. In particular, bioactive glasses have been disclosed comprising SiO₂ at above 54 mol % and Na₂O at 10 mol %. However such glasses require the addition of calcium fluoride for bioactivity. Glasses containing less than 12 mol % Na₂O have been reported U.S. Pat. No. 5,120,340 and EP 0802890, however, these glasses exhibit reduced bioactivity. This is attributable to the fact that glasses with low alkali metal content reported in the art generally contain higher levels of silicon dioxide, which can increase the Network Connectivity and have a detrimental effect on the biological activity of the glass.

In order to increase the suitability of bioactive glasses for in vivo applications, including those discussed above, it is therefore desirable to provide new bioactive glass compositions, for example compositions with lower levels of Na₂O and K₂O and good levels of bioactivity. There is therefore a need in the art for new bioactive glass compositions which provide good levels of bioactivity and which can be formulated and used in a wide range of applications.

In particular, it is an aim of the present application to provide a bioactive glass with enhanced bioactivity. The bioactive glass of the present invention thereby provides an increased rate of apatite deposition and wound healing, leading to rapid repair and reconstruction of diseased and damaged tissues.

The first aspect of the present invention therefore provides a bioactive glass comprising strontium (Sr) and silicon dioxide (SiO₂).

In the context of the present invention, a glass is considered to be bioactive if, on exposure to SBF, deposition of a crystalline HCA layer occurs within three days. In some preferred embodiments, HCA deposition occurs within 24 hours.

Strontium is a bone-seeking trace element which has various effects on bone metabolism. In particular, strontium has been shown to improve vertebral bone density in osteoporotic patients, to increase trabecular bone volume and to increase the extent of bone forming surfaces. However, strontium is provided in the art as a pharmaceutical composition for oral administration and has not previously been incorporated into a bioactive glass, possibly due to a mistaken view that strontium is radioactive.

The inventors have unexpectedly found that incorporation of strontium into a bioactive glass alters the bioactive properties of the glass such that the rate of degradation of the glass and hydroxycarbonated apatite deposition are increased. The bioactive glass of the first aspect is therefore particularly preferred for use in the prevention and/or treatment of damage to tissues such as bone and teeth.

As discussed above, conventional bioactive glasses comprise calcium oxide (CaO). The inventors have found that providing a bioactive glass comprising a source of Sr significantly increases the rate of hydroxycarbonated apatite deposition on the surface of the bioactive glass when it is exposed to body fluid, compared to conventional bioactive glasses. It is proposed that the use of a bioactive glass comprising a source of Sr results in the replacement of a proportion of the Ca²⁺ ions in the resulting hydroxycarbonated apatite, providing a mixed Sr²⁺/Ca²⁺ hydroxycarbonated apatite. This Sr²⁺ substituted hydroxycarbonated apatite has a lower solubility product than unsubstituted hydroxycarbonated apatite, leading to an increase in the rate of hydroxycarbonated apatite deposition. However, a second more important mechanism further increases the rate of hydroxycarbonated apatite deposition. The strontium cation is larger in size than the calcium, having an ionic size of 1.08×10⁻¹⁰ m (compared with 0.99×10⁻¹⁰ m for calcium). Substitution of strontium cations for calcium cations in the bioactive glass results in an expansion of the glass network as a result of the reduced interaction between the strontium atoms and the non-bridging oxygens in the network. This expansion in the bioactive glass network increases the degradability of the bioactive glass, increasing bioactivity and the rate of hydroxycarbonated apatite deposition. Strontium therefore acts as a network modifier, altering the structure of the glass network so as to improve or provide beneficial properties to the glass. The bioactive glass of the first aspect of the invention therefore increases the rate at which the bioactive glass forms a bond with tissues such as bone. Furthermore, the strontium atoms have a direct stimulatory effect on the osteoblasts leading to increased bone formation.

For the purposes of the first aspect of the invention, the bioactive glass comprises a source of strontium, preferably a source of Sr²⁺. The strontium may be provided in the form of strontium oxide (SrO), or as a source of strontium oxide. A source of strontium oxide is any form of strontium which decomposes to form strontium oxide (SrO), including but not limited to strontium carbonate (SrCO₃), strontium nitrate (SrNO₃), strontium acetate (Sr (CH₃CO₂)₂) and strontium sulphate (SrSO₄). The strontium may also be incorporated as strontium fluoride (SrF₂), strontium phosphate (Sr₃(PO₄)₂) and strontium silicate.

The bioactive glass can comprise strontium at a level (molar percentage) of 0.05 to 40%, 0.1 to 40%, more preferably 0.1% to 17%, 0.2% to 17%, more preferably 0.1% to 2% or 0.2% to 2% more preferably 0.3% to 2%, more preferably 0.4% to 1.5%, preferably 6% to 30%, more preferably 7% to 18%, more preferably 8% to 17%, more preferably 10% to 13%.

Thus, preferably, the bioactive glass of the invention comprises a molar percentage of a source of strontium of at least 0.1%, preferably at least 0.2% or at least 2% (for example 0.1-40%, 0.1% to 17% or 0.2-17%, more preferably 0.1% to 2% or 0.2% to 2% more preferably 0.3% to 2% or 0.4% to 1.5%, more preferably 6% to 30%, 7% to 18%, 8% to 17%, or 10% to 13%).

When the strontium is provided as SrO, the molar percentage of SrO in the bioactive glass is preferably 0.2% to 45%. More preferably, the molar percentage of SrO in the bioactive glass is 0.2 to 40%, 0.3% to 40%, 2 to 40%, 3 to 40%, 3 to 25% or 3% to 15%.

The SrO content of the bioactive glass can be used to vary the rate of hydroxycarbonated apatite (HCA) formation. The rate of metabolic tissue repair determines how quickly bonding between the tissue and a bioactive material can progress. Therefore, compatibility between the bioactive material and the surrounding tissue will be maximized when the material's bioactivity rate (the speed with which HCA is produced) matches the body's metabolic repair rate. In particular, it is desirable to match the rate of degradation of the bioactive glass to the rate of tissue ingrowth. However, an individual's repair rate or rate of tissue ingrowth can vary with age and disease state, among other factors, rendering identification of a single, ideal bioactivity rate impossible. It can therefore be highly useful to vary the rate of hydroxycarbonated apatite formation or rate of degradation of the bioactive glass by varying the SrO content of the glass. Increasing replacement of Ca by Sr expands the glass network and accelerates the rate of HCA formation. The rate of hydroxycarbonated apatite formation also depends upon the SiO₂ content of the glass.

The bioactive glass may additionally comprise one or more additional components. The additional components may comprise one or more of calcium, phosphate, magnesium, zinc, boron or fluorine and an alkali metal such as sodium and potassium.

Preferably these components are provided as compounds including but not limited to sodium oxide (Na₂O), sodium carbonate (Na₂CO₃), sodium nitrate (NaNO₃), sodium sulphate (Na₂SO₄), sodium silicates, potassium oxide (K₂O), potassium carbonate (K₂CO₃), potassium nitrate (KNO₃), potassium sulphate (K₂SO₄), potassium silicates, calcium oxide (CaO), calcium carbonate (CaCO₃), calcium nitrate (Ca(NO₃)₂), calcium sulphate (CaSO₄), calcium silicates, magnesium oxide (MgO), magnesium carbonate (MgCO₃), magnesium nitrate (Mg(NO₃)₂), magnesium sulphate (MgSO₄), magnesium silicates, zinc oxide (ZnO), zinc carbonate (ZnCO₃), zinc nitrate (Zn(NO₃)₂), zinc sulphate (ZnSO₄), and zinc silicates and any such compounds, including acetates of sodium, potassium, calcium, magnesium or zinc, that decompose to form an oxide.

It will be appreciated that the exact molar percentage of the components of the bioactive glass affects the physical and biological properties of the bioactive glass. Different uses of the bioactive glass may require different properties, and hence the properties of the bioactive glass may be tailored to a particular intended use by adjusting the molar percentage of each component.

Preferably, the bioactive glass comprises a source of sodium, including but not limited to sodium oxide (Na₂O), sodium carbonate (Na₂CO₃), sodium nitrate (NaNO₃), sodium sulphate (Na₂SO₄) and sodium silicates. Sodium may act as a network modifier within the bioactive glass structure.

Traditionally, the mechanism proposed for the deposition of hydroxycarbonated apatite on bioactive glass relies on the presence of sodium ions. It is understood that sodium ions are exchanged for protons in the external fluid resulting in an alkaline pH. This alkaline pH allows alkaline hydrolysis of Si—O—Si bonds of the glass network. However, recent work by the inventors has shown that sodium ions do not have to be present for the bioactive glass to be bioactive. The desirable level of sodium ions in the bioactive glass depends upon the intended application. As described above, for many applications it is desirable to produce a bioactive glass with low levels of sodium.

In a typical existing bioactive glass, such as 45S5, the molar % of Na₂O is approximately 25%. The inclusion of strontium in the bioactive glass of the present invention allows low molar percentages of sodium (for example Na₂O) to be used, whilst maintaining the bioactivity of the glass. In particular, the replacement of calcium with strontium in the glass of the invention expands the glass network, facilitating the degradation of the glass and increasing bioactivity.

Preferably the bioactive glass comprises a source of sodium ions at a molar percentage of 0-30%, 0-25%, 3 to 25%, 5-25%, 3-15% or 3-6%. Preferably the source of sodium ions is sodium oxide.

Preferably, the bioactive glass comprises a source of potassium including but not limited to potassium oxide (K₂O), potassium carbonate (K₂CO₃), potassium nitrate (KNO₃), potassium sulphate (K₂SO₄) and potassium silicates. As with sodium, the potassium may act as a network modifier within the bioactive glass structure. As described above, it is advantageous to provide a bioactive glass composition in which the potassium content is low.

Preferably the bioactive glass comprises a source of potassium ions at a molar percentage of 0-30%, 0 to 25%, 3 to 25%, 5 to 25%, 0 to 7%, or 3 to 7%. Preferably the source of potassium ions is potassium oxide.

Preferably the combined molar percentage of the source of sodium and potassium is 0-30%. Preferably the combined molar percentage of Na₂O and K₂O in the bioactive glass is 0%-30%. More preferably, the combined molar percentage of the source of sodium and potassium (e.g. of Na₂O and K₂O) in the bioactive glass is 0 to 28% or 5% to 28%. For certain applications, the combined molar percentage of the source of sodium and potassium (for example Na₂O and K₂O) in the bioactive glass is 0 to 15% or 5% to 15%. In certain preferred embodiments, the glass is free from sodium and potassium.

The bioactive glass of the present invention preferably comprises a source of calcium including but not limited to calcium oxide (CaO), calcium carbonate (CaCO₃), calcium nitrate (Ca(NO₃)₂), calcium sulphate (CaSO₄), calcium silicates or a source of calcium oxide. For the purposes of this invention, a source of calcium oxide includes any compound that decomposes to form calcium oxide. Release of Ca²⁺ ions from the surface of the bioactive glass aids the formation of the calcium phosphate-rich layer on the surface of the glass. The provision of calcium ions by the bioactive glass can increase the rate of formation of the calcium phosphate-rich layer. However it should be appreciated that the calcium phosphate-rich layer can form without the provision of calcium ions by the bioactive glass, as body fluid itself contains calcium ions. Thus, for the purposes of this invention, bioactive glasses containing no calcium can be used. Preferably, the molar percentage of Ca is 0% to 50% or 0% to 40%. More preferably, the bioactive glass comprises a source of calcium ions (preferably CaO) at a molar percentage of 0% to 40%, 0 to 30% or 5 to 30%.

The bioactive glass of the present invention preferably comprises P₂O₅. Release of phosphate ions from the surface of the bioactive glass aids in the formation of hydroxycarbonated apatite. Whilst hydroxycarbonated apatite can form without the provision of phosphate ions by the bioactive glass, as body fluid itself contains phosphate ions, the provision of phosphate ions by the bioactive glass increases the rate of formation of hydroxycarbonated apatite. In addition, the provision of P₂O₅ has a beneficial effect on the viscosity-temperature dependence of the glass, increasing the working temperature range, which is advantageous for the manufacture and formation of the glass. Preferably, the molar percentage of P₂O₅ is 0% to 14%. More preferably, the molar percentage of P₂O₅ is 0% to 8%. More preferably, the molar percentage of P₂O₅ is at least 0.5% or 1%, preferably 1% to 2%.

The bioactive glass of the present invention preferably comprises a source of magnesium including but not limited to magnesium oxide (MgO), magnesium carbonate (MgCO₃), magnesium nitrate (Mg(NO₃)₂), magnesium sulphate (MgSO₄), magnesium silicates and any such compounds that decompose to form magnesium oxide. Recent data indicates that magnesium can act partially as an intermediate oxide and partially as a network modifier. Magnesium ions decrease the size of the hydroxycarbonated apatite crystals formed and decrease the thermal expansion coefficient of the glass. This is advantageous when the bioactive glass is intended for use as a coating, for example as a coating on metal prosthesis, including but not limited to those comprising metal alloys such as Ti6Al4V. The ability to decrease the thermal expansion coefficient of the bioactive glass coating allows the thermal expansion coefficient of the coating to be matched to that of the metal prosthesis, preventing debonding of the coating from the substrate during cooling. In particular, the thermal expansion coefficient of the bioactive glass coating can be matched to medical grade alloys used in the art.

Preferably, the molar percentage of the source of magnesium (preferably MgO) is 0% to 20%, 0% to 12%, 2 or 3% to 30%, or 10% to 20%. Preferably, at least 2% of 3% is present. A portion or all of the magnesium can be provided as magnesium oxide. The presence of magnesium oxide acts to suppress apatite crystal size thereby reducing the formation of brittle bone.

The bioactive glass of the present invention preferably comprises a source of zinc, including but not limited to zinc oxide (ZnO), zinc carbonate (ZnCO₃), zinc nitrate (Zn(NO₃)₂), zinc sulphate (ZnSO₄), and zinc silicates and any such compounds that decompose to form zinc oxide. Zinc has not been previously incorporated into bioactive glasses. The inventors have found however that the incorporation of zinc into the bioactive glass of the present invention promotes wound healing and aids the repair and reconstruction of damaged bone tissue. The provision of zinc ions also decreases the size of the hydroxycarbonated apatite crystals formed and decreases the thermal expansion coefficient. This is advantageous when the bioactive glass is intended for use as a coating, as described above. Zinc can also act as a network modifier within the bioactive glass structure. Preferably, the molar percentage of the zinc source (preferably ZnO) is 0% to 10%, 0% to 5%, 0% to 3%. Preferably at least 2% is present, more preferably, 2% to 3% is present.

The bioactive glass of the present invention preferably comprises boron, preferably as B₂O₃. As with P₂O₅, B₂O₃ is believed to have a beneficial effect on the viscosity-temperature dependence of the glass, increasing the working temperature range which is advantageous for the manufacture and formation of the glass. B₂O₃ is also believed to increase the size of the processing window between the glass transition temperature of the bioactive glass and the onset temperature for crystallisation, allowing the sintering of bioactive glass powders without crystallisation. This is advantageous as the formation of crystals in the bioactive glass generally decreases its bioactivity. Preferably, the molar percentage of B₂O₃ is 0% to 15%. More preferably, the molar percentage of B₂O₃ is 0% to 12%, or 0% to 2% Preferably, at least 1% is present.

The bioactive glass of the present invention preferably comprises fluorine. Preferably, fluorine is provided in the form of one or more of calcium fluoride (CaF₂), strontium fluoride (SrF₂), magnesium fluoride (MgF₂), Sodium fluoride (NaF) or potassium fluoride (KF). Fluoride stimulates osteoblasts, and increases the rate of hydroxycarbonated apatite deposition. Fluoride and strontium function synergistically in this regard. Fluoride also promotes the formation of more mixed-type apatite structures with a greater similarity to natural biological forms by substituting readily for hydroxyl ions in the apatite lattice. The mixed apatite is more thermodynamically stable and therefore less soluble and less resorbable. Fluoride can also be used to decrease the melting temperature of the bioactive glass. Preferably, the fluorine is provided in a molar percentage of 0% to 50%, more preferably 0% to 25%. Preferably, the source of fluorine (preferably CaF₂) is provided in a molar percentage of 0% to 10%, or 1% to 7%. Preferably at least 1% is present.

The first aspect of the invention preferably provides a bioactive glass comprising a combined molar percentage of SrO, CaO, MgO, Na₂O and K₂O of 40% to 60%. More preferably, the combined molar percentage of SrO, CaO, MgO, Na₂O and K₂O is 45% to 55%.

In one embodiment the bioactive glass may additionally comprise silver. Preferably the silver is provided as silver oxide. Preferably, the silver is provided in a molar percentage up to 1%, 0.75%, 0.5% or 0.25%. The inclusion of silver can advantageously provide the bioactive glass with antibacterial properties.

Aluminium is a neurotoxin and inhibitor of in vivo bone mineralisation even at very low levels, for example <1 ppm. Therefore, preferably, the bioactive glass of the present invention is aluminium-free.

Preferably, the glass is free of iron-based oxides, such as iron III oxides, e.g. Fe₂O₃, and iron II oxides, e.g. FeO.

The bioactive glass may be provided as, for example, a melt-derived bioactive glass or a sol-gel derived bioactive glass and can be prepared using known melt quench or sol gel techniques. The melt-derived or sol-gel derived bioactive glass can further be sintered using known technology. Both melt-derived and sol gel-derived glasses can comprise one or more of the above-identified additives (sources of Na, K, Ca, P₂O₅, Mg, Zn, B₂O₃, F or Ag).

As stated above, in the first aspect of the present invention the bioactive glass comprises silicon dioxide (SiO₂). The preferred molar percentage of silicon dioxide in the bioactive glass depends in part upon the method of production of the bioactive glass.

Bioactive glass powders can be produced by conventional melt techniques well known in the art. Melt-derived bioactive glass is preferably prepared by mixing and blending grains of the appropriate carbonates or oxides, melting and homogenising the mixture at temperatures of approximately 1250° C. to 1500° C. The mixture is then cooled, preferably by pouring the molten mixture into a suitable liquid such as deionised water, to produce a glass frit.

Melt-derived glasses have a silicate structure which is predominantly Q² in character, i.e. consisting of a silicon with two bridging oxygens linked to two other silicons and two non-bridging oxygens. As stated above, conventional melt-derived bioactive glasses require alkali metal oxides such as Na₂O and K₂O to aid in melting or homogenisation, and the incorporation of such alkali metal oxides has significant disadvantages. However, the incorporation of strontium into melt-derived glasses allows the use of lower concentrations of Na₂O and K₂O, as well as increasing the rate of hydroxycarbonated apatite deposition.

The production of ceramic and glass materials by the sol-gel process has been known for many years and is described in U.S. Pat. No. 5,074,916 and Hench & West, The Sol-Gel Process, 90 Chem. Rev. 33 (1990). The sol-gel process essentially involves mixing of the glass precursors (metal alkoxides in solution) into a sol (a dispersion of colloidal particles in a liquid), followed by hydrolysis, gelation and firing at a temperature of approximately 200-900° C. The mixture is cast in a mould prior to gelation of the mixture, in which the colloidal sol particles link together to form a rigid and porous three-dimensional network which can be aged, dried, chemically stabilised and/or densified to produce structures with ranges of physical properties. All of these steps can be carried out at relatively low temperatures as compared with melt derived processes, typically 600-800° C.

Sol-gel derived bioactive glasses retain their bioactive properties with higher molar percentages of SiO₂ than do melt-derived glasses. As discussed in U.S. Pat. No. 5,074,916, this is thought to be due to the presence of small pores (approximately 1.2 to 20 nm) and large surface area of the sol-gel derived powders, which give rise to a large area density of nucleation sites for hydroxyapatite crystallites, allowing build up of a hydroxyapatite layer to take place at higher rates, with lower proportional concentrations of CaO and P₂O₅ and higher levels of SiO₂ than would be necessary for known melt-derived bioactive glass compositions. For sol-gel derived bioactive glasses of the present invention, the diameter of the pores is preferably 1.2 to 10 nm, and the surface area is preferably at least 40 m²/g.

The process for the production of the bioactive glass of the present invention, whether melt-derived or sol-gel, will therefore affect the molar percentage of SiO₂ that may be used, whilst still maintaining bioactivity.

SiO₂ forms the amorphous network of the bioactive glass, and the molar percentage of SiO₂ in the glass affects its Network Connectivity (NC). Network Connectivity is the average number of bridging bonds per network forming element in the glass structure. NC determines glass properties such as viscosity, crystallisation rate and degradability. At a NC of 2.0 it is thought that linear silicate chains exist of infinite molar mass. As NC falls below 2.0, there is a rapid decrease in molar mass and the length of the silicate chains. At an NC above 2.0, the glass becomes a three dimensional network.

For melt-derived glasses to be bioactive, NC must be below 2.6, or more preferably below 2.4. The bioactive glass of the first aspect therefore has a network connectivity of 2.6 or less, preferably 2.4 or less.

Preferably, the molar percentage of SiO₂ in a melt-derived bioactive glass is 30% to 60%. More preferably, the molar percentage of SiO₂ in a melt-derived bioactive glass is 40% to 57%

In a preferred embodiment of the first aspect, the combined molar percentage of SiO₂, P₂O₅, and B₂O₃ in a melt-derived bioactive glass does not exceed 60%. At values higher than 60%, the network connectivity of a melt derived bioactive glass is unfavourably high, resulting in an unfavourably low level of bioactivity.

Preferably, the molar percentage of SiO₂ in a sol gel-derived bioactive glass is 50% to 95%. More preferably, the molar percentage of SiO₂ in the sol-gel derived bioactive glass is 60% to 94% or 60 to 86% or 70 to 86%.

Where a bioactive glass of the invention is sol-gel derived and comprises additives as described above (a source of Na, K, Ca, P₂O₅, Mg, Zn, B₂O₃, F or Ag), it is preferable to use a soluble form of the additive, for example a nitrate or acetate.

By varying the SiO₂ content, a range of hydroxycarbonated apatite deposition rates can be obtained. Conversely, varying the time of exposure to actual or simulated in vivo solutions permits the use of a range of allowable proportions of SiO₂.

In a preferred embodiment of the invention, the bioactive glass is a sol gel-derived glass, the composition of which is alkali-metal free.

Depending upon its intended use, the bioactive glass of the first aspect may be in particulate form, or may comprise a solid such as a disk or monolith. In particular, the glass can be provided in any required shape or form, for example as a pellet, sheet, disk, foam, fibre etc.

In some embodiments, the composition of a bioactive glass of the present invention is tailored to provide the glass with a large processing window, resulting from a large gap between the Glass Transition Temperatures (T_{g}) and the onset temperature for crystallisation (T_{c}). Such glasses are particularly suitable for drawing into fibres and for sintering because the large processing window allows processing (for example drawing of the glass into fibres) to be carried out whilst crystallisation is inhibited.

In particulate form, the preferred particle size depends upon the application of the bioactive glass in question, however preferred ranges of particle sizes are less than 1200 microns, preferably between 1 and 1000 microns, more preferably 50 to 800 microns, more preferably 100 to 700 microns. As a general rule, the particle size of sol-gel-derived glasses can be smaller than that of melt-derived glasses. The range of particle size required also depends upon the application and the bioactivity of the glass. For example, fillers for composites or for sintered bioactive glasses would be provided with a particle size of 45 microns or less. Glass particles for use in coatings may be provided with a particle size of less than 38 microns and a mean particle size of 5-6 microns. In particulate form, such as a powder, the bioactive glass may be included in a cement, a paste or a composite. The bioactive glass may be included (for example as a filler) in substances including but not limited to acrylic, bisphenol A diglycidylether methacrylate (Bis GMA) and polylactide. The bioactive glass powder may be sintered to create bioactive coatings or to form a porous solid for use as a scaffold. In addition, the bioactive glass may be incorporated into a degradable polymer scaffold. The bioactive glass may be in the form of granules.

The second aspect of the present invention provides a process for the production of the bioactive glass of the present invention, comprising admixing Sr and SiO₂, and optionally one or more of Na, K, Ca, P₂O₅, Mg, Zn, B₂O₃ or F. The process for the production of the bioactive glass of the present invention may be a melt quench method or a sol gel method, as described above and using techniques known in the art.

The third aspect of the invention relates to the bioactive glass of the first aspect of the invention for use in medicine, preferably for use in the prevention and/or treatment of damage to a tissue.

For the purposes of this invention, the tissue can be bone tissue, cartilage, soft tissues including connective tissues and dental tissues including calcified dental tissues such as enamel and dentin.

The tissues of the third aspect can be animal tissues, more preferably mammalian or human tissues. The bioactive glass of the third aspect is therefore preferably provided for use in humans or animals such as dogs, cats, horses, sheep, cows or pigs.

Throughout this text, the prevention and/or treatment means any effect which mitigates any damage or any medical disorder, to any extent, and includes prevention and treatment of damage itself as well as the control of damage. The term “treatment” means any amelioration of disorder, disease, syndrome, condition, pain or a combination of one or more thereof. The term “control” means to prevent the condition from deteriorating or getting worse for example by halting the progress of the disease without necessary ameliorating the condition. The term “prevention” means causing the condition not to occur, or delaying the onset of a condition, or reducing the severity of the onset of the condition.

In particular, the terms prevention and/or treatment include the repair and/or reconstruction of tissue. For the purposes of this invention, the term “repair” means the restoration of the tissue to a condition of working order for example by the in vivo stimulation of biological processes. The term “reconstruction” means the rebuilding of the tissue and includes the temporary or permanent incorporation into the tissue of an external component such as a scaffold, model etc.

The bioactive glass of the third aspect is provided to prevent or treat damage to tissues. For the purposes of this invention the damage can be mechanical damage, can be caused by an external agent or can be a result of an internal biological process. Examples of mechanical damage include damage caused by trauma, surgery, age related wear, etc. Examples of damage caused by an external agent include damage caused by a medicament, a toxin, or a treatment regime (such as chemotherapy or radiotherapy), for example dialysis-related amyloidosis, damage caused by diseases such as a bacterial, viral or fungal infection, such as osteomyelitis, a genetic condition such as osteogenesis imperfecta and hypophosphotasia, inadequate nutrition, age-related disorders, a degenerative disorder or condition such as osteoporosis and bone cancers including osteosarcoma and Ewing's sarcoma. Examples of damage caused as a result of an internal biological process include an autoimmune disease.

In particular, the damage to the tissue may be caused by or may be a result of osteoarthrosis, periodontal disease, etc.

Release of Sr²⁺ from bioactive glass allows a localised, targeted release of strontium to those areas that require it. This is particularly useful where the bioactive glass is being applied to damaged tissue that would benefit from a localised increase in the deposition of HCA, for example in the treatment of osteoporotic bone. In this respect the bioactive glass of the present invention has a particular advantage over orally-administered pharmaceutical compositions comprising strontium. The rate of release of Sr²⁺ from the bioactive glass can be controlled by modifying the bioactive glass composition or surface area. Both melt-derived glasses and sol-gel derived glasses can be used for localised, targeted release of Sr²⁺.

The provision of bioactive glass of third aspect allows the repair and reconstruction of damaged tissues. In particular, it is submitted that emersion of the bioactive glass in body fluid results in the formation of a HCA layer at the required site of action and the activation of in vivo mechanisms of tissue regeneration. It is proposed that application of the bioactive glass to damaged tissues stimulates the deposition of HCA on the bioactive glass and the surrounding tissues. The bioactive glass of the third aspect therefore causes repair of damaged tissue by initiating and/or stimulating deposition of HCA thereby initiating and/or stimulating regeneration of the damaged tissue.

The bioactive glass of the third aspect may be provided to prevent and/or treat damage by the initiation and/or stimulation of tissue repair without incorporation of the bioactive glass into the tissue. Alternatively or in addition, the bioactive glass may become incorporated into the tissue, such incorporation of the bioactive glass allowing the reconstitution of the tissue. The incorporation of the bioactive glass into the tissue may be permanent or temporary. To this end, the bioactive glass of the third aspect may be used to form a bioactive coating on implants such as prostheses. The bioactive coating allows the formation of a HCA layer between the implant and the surrounding tissue, and effectively binds the implant to the surrounding tissue. Alternatively, the bioactive glass itself may be used as a bone substitute or for extending bone autograft.

The bioactive glass of the third aspect can be used to promote bone formation. More preferably, the bioactive glass is used to increase the rate of apatite deposition, resulting in bone formation. The bioactive glass can be used to repair fractures such as bone fractures. In particular, the bioactive glass is used in Fracture Fixation Devices such as plates screws, pins and nails. The bioactive glass stimulates the deposition of HCA and the formation of bone in and around the site of the fracture.

The bioactive glass of the third aspect can be used to treat damage to tissues in the dental cavity. In a preferred feature of the third aspect of the present invention, the bioactive glass is used for the treatment of periodontal disease. In particular, the bioactive glass is used to promote HCA deposition and bone formation at sites where periodontal disease has resulted in the destruction of the bone that supports the tooth. The bioactive glass can be used further to prevent or treat tooth cavities. Preferably, the bioactive glass is used as a filler to treat tooth cavities and/or to prevent further deterioration of the tooth. The formation of the HCA layer on the surface of the bioactive glass allows the formation of a strong bond between the bioactive glass and calcified tooth tissues such as calcified tooth chop tissues, including enamel and bone. The bioactive glass can be used further to promote tooth mineralization (deposition of hydroxycarbonated apatite), as saliva has a similar ionic composition to that of body fluid. The bioactive glass can be used as a filler in dental composites such as Bis glycidyldimethacrylate and related resins in order to promote apatite formation and inhibit loss of tooth mineral, thereby preventing dental caries. The bioactive glass can be used to treat hypersensitivity in teeth. More preferably, the bioactive glass is used to increase the rate of HCA deposition, resulting in surface occlusion of the dentinal tubules. Such bioactive glass may, for example, be incorporated into toothpastes, dentrifices, chewing gums or mouth washes.

In a preferred feature of the third aspect of the present invention, the bioactive glass is used for vertebroplasty or kyphnoplasty. The bioactive glass may be incorporated into a polymer or cement and injected into the vertebral space by a minimally invasive surgery procedure to prevent osteoporotic fractures and vertebral collapse associated with osteoporosis and resulting in curvature of the spine or to restore height to the vertebrae.

Administration of the bioactive glass results in an increase in pH at the site of action of the bioactive glass due to physiochemical reactions on the surface of the bioactive glass. Bacteria found on the surface of the human skin which thrive under acid conditions are inhibited by the alkaline conditions produced by the bioactive glass. In addition, Sr²⁺ inhibits bacteria, including but not limited to Staphylococcus aureus, Streptococcus mutans and Actinomyces viscosus.

In a preferred feature of the third aspect of the invention, the bioactive glass of the third aspect is therefore provided for the prevention and/or treatment of a bacterial infection associated with damage to a tissue. Preferably, the bacterial infection is caused by Staphylococcus aureus.

The fourth aspect of the present invention provides a coating comprising a bioactive glass of the first aspect of the invention.

The coating can be used to coat implants for insertion into the body, combining the excellent mechanical strength of implant materials such as metal and metal alloys such as Ti6Al4V and chrome cobalt alloys, plastic and ceramic, and the biocompatibility of the bioactive glass. The bioactive glass coating can be applied to the metal implant surface by methods including but not limited to enamelling or glazing, flame spraying, plasma spraying, rapid immersion in molten glass, dipping into a slurry of glass particles in a solvent with a polymer binder, or electrophoretic deposition. For example, prosthetics comprising the metal alloy Ti6Al4V can be coated with a bioactive glass by plasma spraying, with or without the application of a bond coat layer.

The bioactive coating allows the formation of a hydroxycarbonated apatite layer on the surface of the prosthesis, which can support bone ingrowth and osseointegration. This allows the formation of an interfacial bond between the surface of the implant and the adjoining tissue. The prosthesis is preferably provided to replace a bone or joint such as comprise hip, jaw, shoulder, elbow or knee prostheses. The prostheses of the fourth aspect provided can be for use in joint replacement surgery. The bioactive coating of the fourth aspect of the present invention can also be used to coat orthopedic devices such as the femoral component of total hip arthroplasties or bone screws or nails in fracture fixation devices.

The incorporation of magnesium ions and zinc ions into the bioactive glass of the present invention decreases the thermal expansion coefficient, which is advantageous when the bioactive glass is intended for use as a coating. Magnesium ions and zinc ions increase TEC but decrease it when substituted for CaO or SrO. The ability to decrease the thermal expansion coefficient of the bioactive glass coating allows the thermal expansion coefficient of the coating to be matched to that of the prosthesis, preventing cracking of the coating during cooling.

Thus, bioactive glass for use as a coating preferably comprises multiple components, including magnesium and zinc ions. A multicomponent composition acts to increase the entropy of mixing and avoid the stoichiometry of known crystal phases, in order to promote sintering without crystallisation occurring. The optimum sintering temperature can be obtained by performing Differential Scanning Calorimetry over a range of heating rates and extrapolating the onset temperature for crystallization to zero heating rate. The greater the temperature difference between the glass transition temperature and the extrapolated crystallization onset temperature, the larger the processing window.

Preferably, the bioactive glass of the present invention may be provided as a coating for Ti6Al4V or for Chrome Cobalt alloys. Preferably the coating is put down on the alloy at a temperature below the crystallisation temperature onset. Preferably the bioactive glass for the coating is sintered to full density, and has a predominantly Q² silicate structure in order to ensure bioactivity.

The coating of the present invention may comprise one or more layers of the bioactive glass of the present invention. For example a single layer coating or a bilayer coating may be provided. The one or more layers of the coating may all comprise bioactive glass of the present invention. Alternatively, the coating may be a bilayer or multi-layer coating in which at least one of the layers comprises a Sr-containing bioactive glass of the first aspect of the invention and at least one layer does not comprise a Sr-containing bioactive glass. A bilayer coating for use with chrome cobalt alloys preferably comprises a base layer which is chemically stable and non-bioactive, and one or more top layers comprising a bioactive glass according to the present invention.

A bilayer coating may comprise two layers of bioactive glass. For example, it may be preferable to provide a less bioactive and more chemically stable base layer and a more bioactive and less chemically stable top layer. The more reactive top layer will allow optimum bioactivity to promote osseointegration, whilst the less reactive base layer will ensure that the prosthesis remains coated for a long period of time in the body. Both layers may comprise bioactive glasses of the present invention. Alternatively, a bilayer could be provided wherein the base layer comprises a less reactive bioactive glass, for example a glass known in the art, which does not comprise strontium, and wherein the top layer comprises a more bioactive glass of the present invention.

Bilayer coatings may also be provided to prevent dissolution of ions from the prosthesis into the surrounding fluid and/or tissue. Bilayer coatings on chrome cobalt are particularly desirable since there can be significant dissolution of the oxides of cobalt, nickel and chromium from the protective oxide layer into the glass which could then be released from the glass. For this reason a chemically stable base coating glass composition is preferred.

Single layer coatings may be fabricated using a process as described in Example 6. Bilayer coatings may be fabricated using a two step process, for example as described in Examples 7 and 8. Preferably, the coating is between 50 and 300 microns thick.

The bioactive glass for use as a coating preferably comprises approximately 49%-50% SiO₂, approximately 0.5% to 1.5%% P₂O₅, approximately 8% to 30% % CaO, approximately 8% to 17% SrO, approximately 3 to 7% Na₂O, approximately 3 to 7% K₂O, approximately 3% ZnO, approximately 7 to 16% MgO and approximately 0 to 6% CaF₂. More preferably, the coating comprises a bioactive glass comprising approximately 50% SiO₂, approximately 1% P₂O₅, approximately 9% to 29% CaO, approximately 9% to 16% SrO, approximately 3 to 7% Na₂O, approximately 3 to 7% K₂O, approximately 3% ZnO, approximately 7 to 16% MgO and approximately 0 to 6% CaF₂.

The fifth aspect of the present invention provides a surgical device comprising the bioactive glass of the first aspect of the invention. In particular, the surgical device is provided for insertion into the body, more particularly for insertion at the site of damage to the tissue, wherein the insertion can be permanent or temporary. The surgical device is particularly provided for use in the prevention and/or treatment of damage to tissues.

In particular, the fifth aspect provides a bioactive porous scaffold comprising a bioactive glass of the first aspect. Preferably, the bioactive porous scaffold is for use in tissue engineering. The porous scaffolds can be used for in vitro synthesis of bone tissue when exposed to a tissue culture medium and inoculated with cells. The bioactive properties of such scaffolds allow the formation of a strong interface between the bone tissue and the scaffold, and the induction of osteoblast proliferation. Amongst other uses, the bone tissue formed on the bioactive porous scaffold can be inserted into areas that exhibit increased risk of fracture, and decreased or even extinct potential for bone tissue formation. In particular, the bone tissue can be used to replace damaged or diseased bone.

The sixth aspect of the present invention provides the bioactive glass of the present invention for use in the prevention and treatment of body odour. More preferably, the bioactive glass is for use as, or in, a deodorant. It is submitted that the bioactive glass increases the pH of the surrounding skin and releases Sr²⁺, wherein the increase in pH and the release of Sr²⁺ have a bactericidal action against the bacteria responsible for the production of body odour.

The seventh aspect of the present invention provides a composition comprising the bioactive glass of the first aspect of the invention. The composition is preferably provided for the prevention and/or treatment of damage to tissue.

The composition of the seventh aspect of the present invention may comprise bioactive glass in the form of bioactive glass particles. The bioactive glass particles may be provided alone, or in combination with additional materials, including but not limited to antibiotics such as erythromycin and tetracycline, antivirals such as acyclovir and gancyclovir, healing promotion agents, anti-inflammatory agents such as corticosteroids and hydrocortisone, immunosupressants, growth factors such as basic fibroblast growth factor, platelet derived growth factor, bone morphogenic proteins, parathyroid hormone, growth hormone and insulin-like growth factor I, anti-metabolites, anti-catabolic agents such as zoledronic acid, bisphosphonates, cell adhesion molecules, bone morphogenic proteins, vascularising agents, anti-coagulants and topical anaesthetics such as benzocaine and lidocaine, peptides, proteins, polymer or polysaccharide conjugated peptides, polymer or polysaccharide conjugated proteins or modular peptides.

The composition of the seventh aspect of the invention may comprise bioactive glass in the form of bioactive glass fibres. Such bioactive glass fibres may be used, for example, to promote soft tissue repair, wherein the soft tissue may comprise, for example, ligaments.

The composition of the seventh aspect of the invention may be a vehicle for delivery of a therapeutic agent selected from the additional material listed above.

In a preferred feature, the composition is incorporated into implanted materials including but not limited to prosthetic implants, stents and plates, to impart anti-bacterial and anti-inflammatory properties to the materials.

In an additional preferred feature, the composition may comprise a composition for topical application, for example, to treat a wound or burn, for use in skin grafting, in which the composition is applied to a graft site prior to application of the donor tissue, or applied to the donor tissue itself, or for use in surgery, applied to a surgical site to minimise post-surgical adhesions, inflammation and infection at the site.

In a preferred feature, the composition is bone cement comprising the bioactive glass of the first aspect. Preferably, the bioactive glass is provided in combination with acrylic. Preferably, the bone cement is for use in the repair and reconstruction of damaged bone tissue. More preferably, the bone cement is used for securing implants, anchoring artificial members of joints, in restoration surgery of the skull and for joining vertebrae. More preferably, the bone cement is for use in vertebroplasty, wherein the bone cement promotes bone formation. Preferably, the bone cement is used in the formation of bone replacement parts. Bone replacement parts include but are not limited to the auricular frame of the outer ear, the incus, malleus and stapes of the middle ear, cranial bones, the larynx and the hard palate. The bone replacement parts may be produced intra-operatively or may be industrially pre-fabricated. The bone cement may additionally contain stabilisers, disinfectants, pigments, X-ray contrast media and other fillers.

The seventh aspect of the present invention additionally or alternatively provides a bone substitute comprising the bioactive glass of the first aspect of the invention. Preferably, the bone substitute is for use in the prevention and/or treatment, more preferably repair or reconstruction of damaged tissues.

The seventh aspect of the present invention additionally or alternatively provides a powder or monolith including a porous scaffold for extending bone autograft comprising a bioactive glass of the first aspect. Bone autografts involve the placement of healthy bone, taken from the patient, into spaces between or around broken bone (fractures) or holes (defects) in the bone. This is advantageous due to the limited amount of bone stock available for transplantation.

The seventh aspect of the present invention additionally or alternatively provides a degradable polymer composite comprising the bioactive glass of the first aspect of the invention. Preferably the bioactive glass is used in combination with polylactide used in the manufacture of the degradable polymer composite. The degradable polymer composite is provided for use in the prevention and/or treatment of fractures, more preferably in the prevention and/or treatment of bone fractures.

The bioactive glass of the present invention can be provided as a filler in a degradable polyester. In particular, the bioactive glass can be provided as a filler in a polylactide or polyglycolide or a copolymer thereof. The bioactive glass thus provides a bioactive component for bone screws, fraction fixation plates, porous scaffolds, etc. The use of the bioactive glass of the present invention is particularly favoured for use as a filler in a degradable polyester as the bioactive glass prevents autocatalytic degradation which is a feature of polyesters currently known in the art. Autocatalytic degradation occurs as the hydrolysis of an ester results in the formation of an alcohol and an acid. As the hydrolysis of an ester is acid catalysed, the generation of an acid causes a positive feedback situation.

Alternatively or additionally the seventh aspect of the present invention provides a dental composite comprising bioactive glass of the first aspect of the invention. Preferably, the bioactive glass is provided in combination with bisphenol A diglycidylether methacrylate (Bis GMA). The dental composite of the seventh aspect is provided for the prevention and/or treatment of damaged tissues, wherein the damaged tissue preferably comprises dental tissue, more preferably calcified dental tissues such as enamel and dentin. More preferably, the dental composite of the seventh aspect is provided for the prevention and/or treatment of tooth cavities. Preferably, the dental composite is used to fill tooth cavities.

The seventh aspect of the present invention additionally or alternatively provides a toothpaste comprising the bioactive glass of the first aspect. Preferably, the toothpaste prevents and/or treats dental cavies, in particular by promoting tooth mineralization through increased hydroxycarbonated apatite deposition. Preferably, the toothpaste treats or prevents hypersensitivity. More preferably, the toothpaste results in the surface occlusion of dentinal tubules by hydroxycarbonated apatite.

The seventh aspect of the present invention additionally or alternatively provides a deodorant comprising the bioactive glass of the first aspect of the present invention. Preferably, the deodorant is for use in the prevention and treatment of body odour.

The seventh aspect of the invention provides an implant material and/or a material for peridontal treatment comprising a bioactive glass of the first aspect of the invention. The bioactive glass preferably comprises from approximately 46 to 50% SiO₂, approximately 0.5% to 1.5% (preferably approximately 1%) P₂O₅, approximately 0 to 2% B₂O₃, approximately 0 to 23% CaO, approximately 0.5 to 24% (preferably 2 to 24%) SrO, approximately 6% to 27% (preferably 7 to 27%) Na₂O, approximately 0 to 13% K₂O, approximately 0 to 2% ZnO, approximately 0 to 2% MgO and approximately 0 to 7% CaF₂.

The seventh aspect of the invention provides a porous sintered scaffold comprising a bioactive glass of the first aspect of the invention. The bioactive glass preferably comprises from approximately 47 to 50% SiO₂, approximately 0.5% to 1.5% (preferably approximately 1%) P₂O₅, approximately 0 to 2% B₂O₃, approximately 8 to 27% CaO, approximately 3 to 15% SrO, approximately 5 to 7% Na₂O, approximately 4 to 7% K₂O, approximately 3% ZnO, approximately 3% MgO and approximately 0 to 9% CaF₂.

The seventh aspect of the invention provides a filler for a composite comprising a bioactive glass of the first aspect of the invention. The bioactive glass preferably comprises from approximately 50% SiO₂, approximately 0.5% to 1.5%% (preferably approximately 1%) P₂O₅, approximately 19 to 22% CaO, approximately 19 to 22% SrO, approximately 3 to 7% Na₂O, approximately 0 to 3% K₂O, approximately 0 to 2% ZnO and approximately 0 to 2% MgO.

The seventh aspect of the invention provides a filler for dental tooth filling comprising a bioactive glass of the first aspect of the invention. The bioactive glass preferably comprises from approximately 50% SiO₂, approximately 0.5% to 1.5%% (preferably approximately 1%) P₂O₅, approximately 10% CaO, approximately 19% SrO, approximately 3% Na₂O, approximately 3% K₂O, approximately 2% ZnO, approximately 2% MgO and approximately 10% CaF₂.

The seventh aspect of the invention provides a polyacid cement comprising a bioactive glass of the first aspect of the invention. The bioactive glass preferably comprises from approximately 49 to 54% SiO₂, approximately 0 to 0.5% to 1.5%% (preferably approximately 1%) P₂O₅, approximately 7 to 10% CaO, approximately 8 to 19% SrO, approximately 7% Na₂O, approximately 3% ZnO and approximately 10 to 20% MgO.

The seventh aspect of the invention provides a toothpaste or a deodorant comprising a bioactive glass of the first aspect of the invention. The bioactive glass preferably comprises from approximately 50% SiO₂, approximately 0.5% to 1.5% (preferably approximately 1%) P₂O₅, approximately 16 to 20% SrO, approximately 26% Na₂O, approximately 3% ZnO and approximately 0 to 4% CaF₂

Alternatively, when the seventh aspect of the invention provide a tooth paste comprising a bioactive glass of the first aspect of the invention, the bioactive glass comprises from approximately 50% SiO₂, approximately 0.5% to 1.5% (preferably approximately 1%) P₂O₅, approximately 16% SrO, approximately 26% Na₂O, approximately 3% ZnO, and approximately 4% CaF₂.

The eighth aspect of the present invention provides a method for the prevention and/or treatment of damage to tissue comprising administering a bioactive glass as defined in the first aspect of the invention to a patient in need of such treatment. Preferably, the tissue comprises bone or dental tissue, including calcified dental tissues such as enamel and dentin. More preferably, the present invention provides the treatment of bone fractures, dental cavities, periodontal disease, hypersensitive teeth, and/or demineralised teeth.

The bioactive glass of the present invention may be administered by any convenient method. The bioactive glass may be administered topically. Examples of topical application include the administration of a cream, lotion, ointment, powder, gel or paste to the body, for example to the teeth or skin. In particular, the bioactive glass can be provided as a toothpaste comprising the bioactive glass for administration to the teeth of a patient suffering from dental cavies, periodontal disease, hypersensitive teeth, etc.

The bioactive glass may be administered surgically or parenterally. Examples of surgical or parenteral administration would include the administration of the bioactive glass into a tissue, by insertion of the device by injection or by a surgical procedure such as implantation, tissue replacement, tissue reconstruction, etc. In particular, the bioactive glass can be introduced into a bone fracture or a damaged region of bone.

The bioactive glass can also be administered orally. For oral administration, the composition can be formulated as a liquid or solid, for example solutions, syrups, suspensions or emulsions, tablet, capsules and lozenges. Administration of the bioactive glass by oral or parental administration may provide the bioactive glass directly at its required site of action. Alternatively, the bioactive glass can be delivered to its site of action, for example by using the systemic circulation. The bioactive glass can be administrated orally, for example to a patient requiring the prevention and/or treatment of damage to the alimentary canal.

All preferred features of each of the aspects of the invention apply to all other aspects mutatis mutandis.

The invention may be put into practice in various ways and a number of specific embodiments will be described by way of example to illustrate the invention with reference to the accompanying drawings, in which:

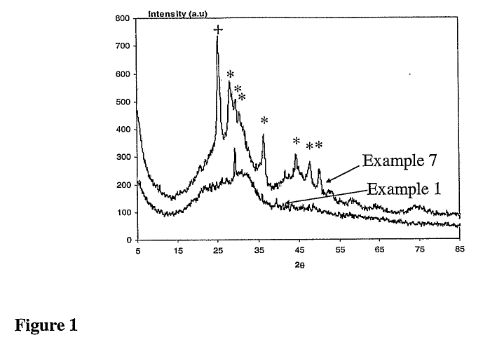

FIG. 1 shows an X-ray diffraction pattern of glasses 1 and 7 as set out in Table 1 (a bioactive glass with and without Sr) after immersion in SBF for 480 mins. The lower trace is glass 1 and the upper trace is glass 7. Peaks marked by ‘*’ are diffraction lines matching HCA. The HCA formation is more pronounced with the strontium-containing glass. The strontium-containing glass also precipitates calcium carbonate (peak marked ‘+’), once all the phosphate in the SBF has been used to form HCA;

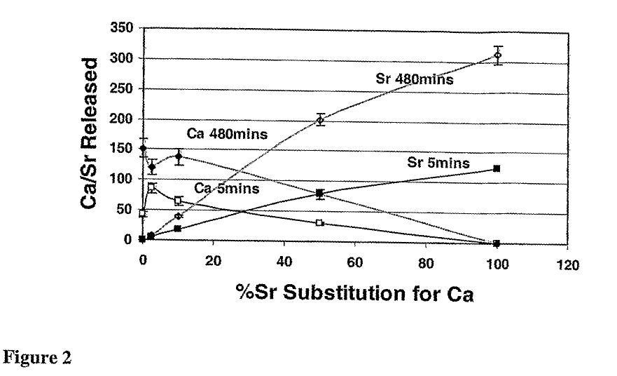

FIG. 2 shows ppm strontium and calcium release from 0.075 g glass samples into 50 ml Tris Buffer pH7.4 at 37° C. after 5 mins and 480 mins for five different glass samples (Examples 1, 2, 3, 5 and 7 as shown in Table 1), which correspond to 0, 2.5, 10, 50 and 100% substitution of Ca by Sr.

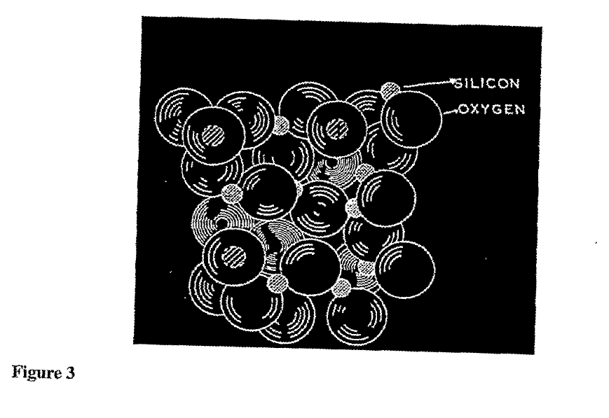

FIG. 3 shows a proposed model for a silica network;

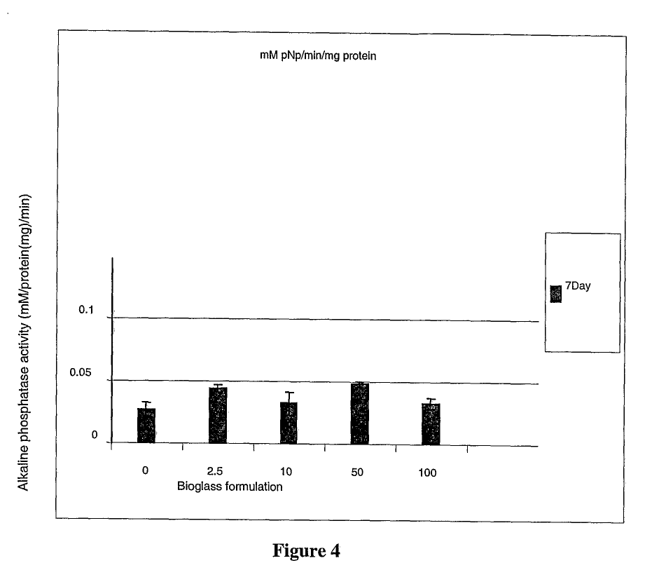

FIG. 4 shows the phosphatase activity (pNp/min) of cells incubated with a bioactive glass comprising 0, 2.5%, 10%, 50% or 100% strontium (Examples 1, 2, 3, 5 and 7 as shown in Table 1, normalised to total protein (mg) after a 7 day period);

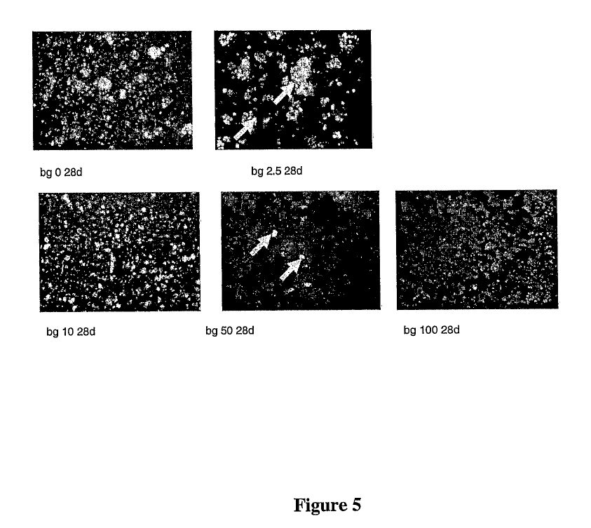

FIG. 5 shows mineralization of cells grown on bioactive glass comprising 0, 2.5%, 10%, 50% or 100% strontium (Examples 1, 2, 3, 5 and 7 as shown in Table 1) for 28 days.

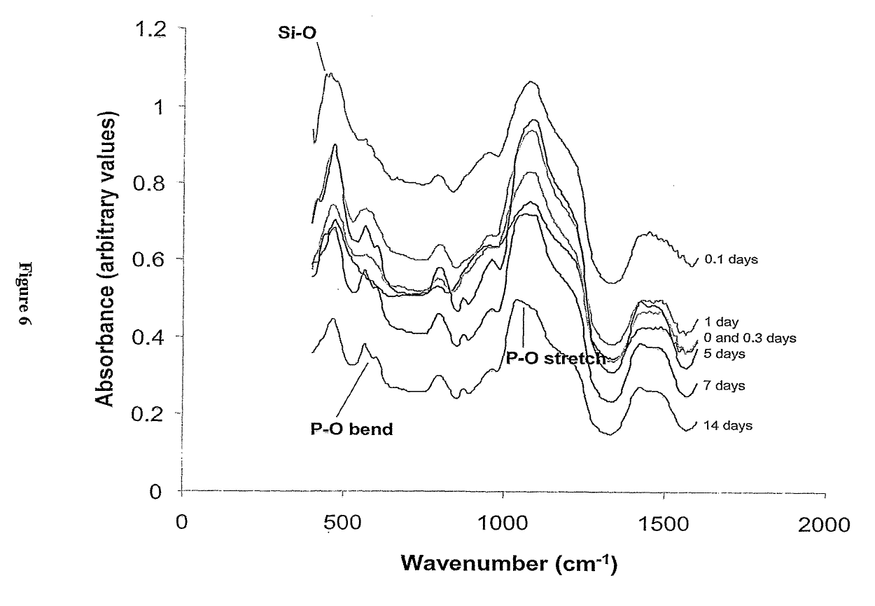

FIG. 6 shows a series of FTIR spectra of glass 7 as shown in Table 1 after incubation in SBF for time periods between 0 and 480 minutes. The lowest trace represents unreacted glass and moving up FIG. 6, the traces represent glass reacted for 5, 15, 30, 60, 120, 240 and 480 minutes respectively.

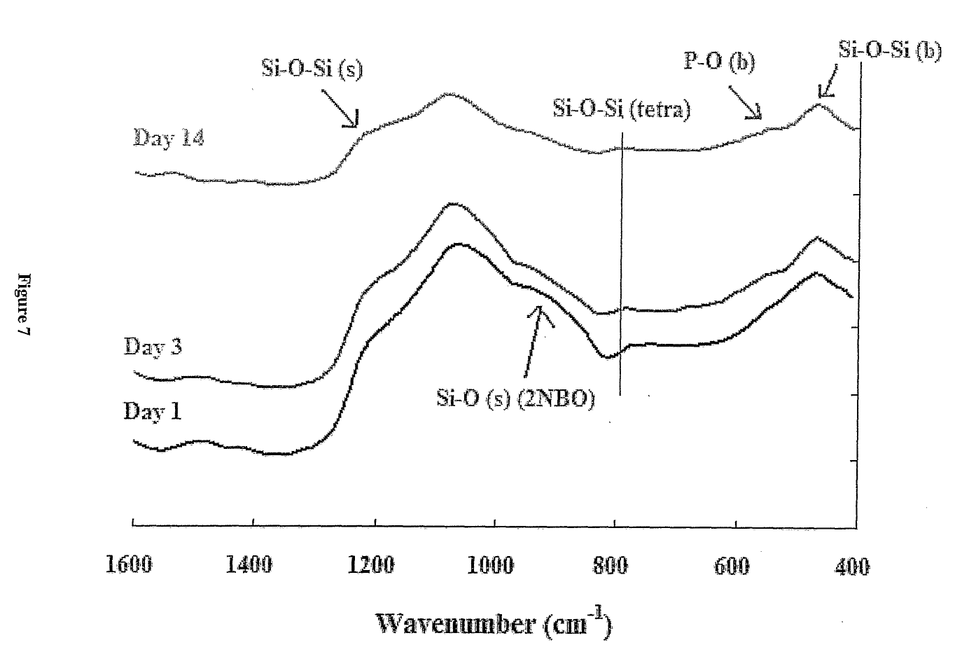

FIG. 7 shows a series of FTIR spectra of glass 12 as shown in Table 1 after incubation in SBF for 0, 0.1, 0.3, 1, 5, 7 and 14 days.

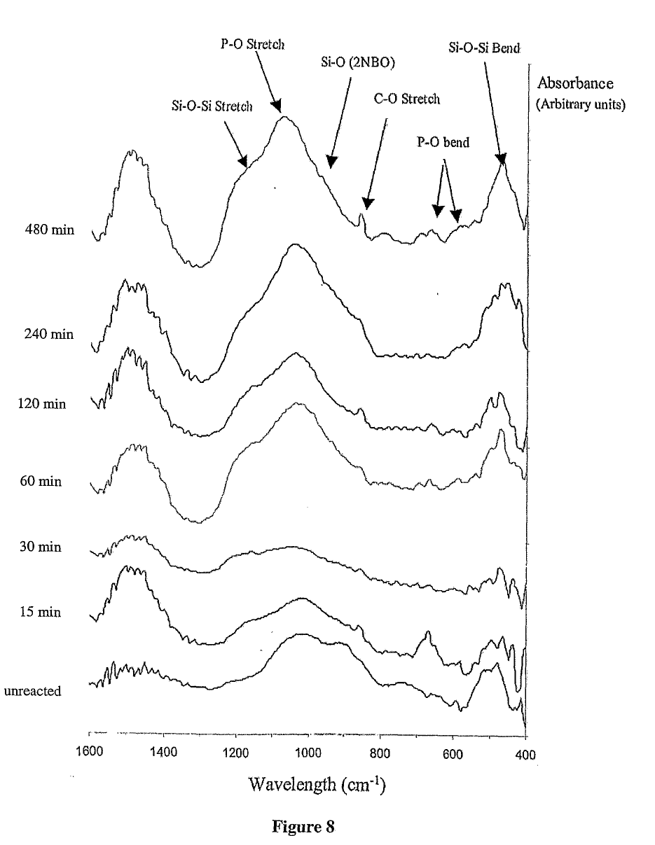

FIG. 8 shows a series of FTIR spectra of glass 29 as shown in Table 1 after incubation in SBF for 1, 3, 7 and 14 days.

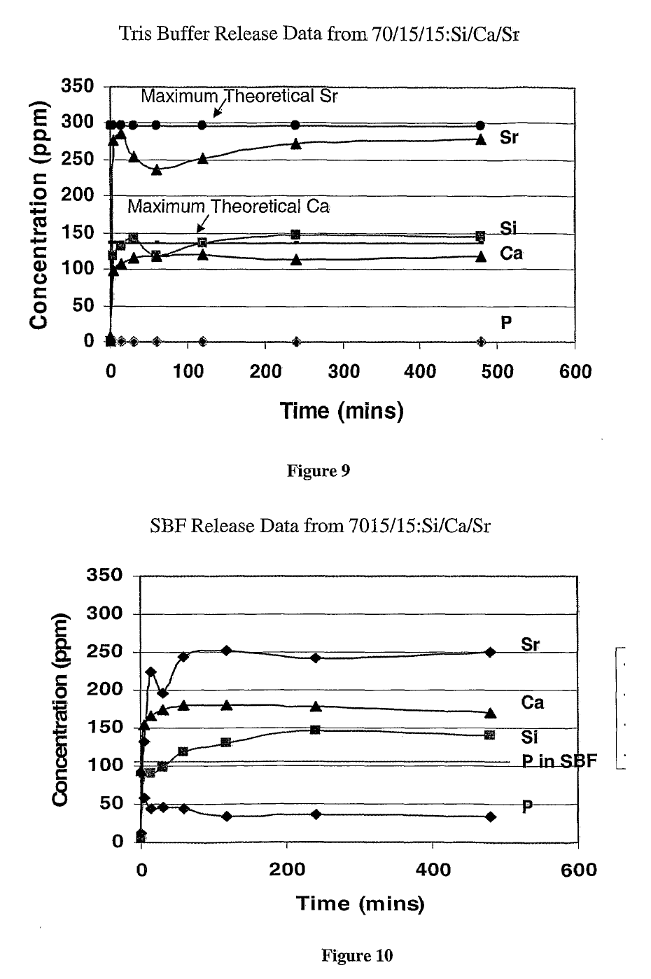

FIGS. 9 and 10 show the results of Tris-Buffer and SBF dissolution assays carried out for glass 43 as shown in Table 4.

The invention will now be illustrated with reference to one or more of the following non-limiting examples.

### EXAMPLES

Tests used in order to determine glass properties are described below.

Throughout the examples set out below, molar percentage values were calculated in accordance with standard practice in the art.

### Dissolution Studies

0.075 mg of <45 μm glass powder was immersed in 50 ml of solution (water, Tris-buffer or SBF) at pH 7.25 and placed in an orbital shaker at 1 Hz for time periods of 5, 15, 30, 60, 120, 240 and 480 min unless otherwise specified. The filtered solution was then analysed by inductively coupled plasma spectroscopy (ICP) to determine the silicon, calcium, sodium and potassium concentration.

### Preparation of Tris-Buffer Solution

For the making of tris-hydroxy methyl amino methane buffer, a standard preparation procedure was taken from USBiomaterials Corporation (SOP-006). 7.545 g of TRAM is transferred into a graduated flask filled with approximately 400 ml of deionised water. Once the THAM dissolved, 22.1 ml of 2N HCl is added to the flask, which is then made up to 1000 ml with deionised water and adjusted to pH 7.25 at 37° C.

### Preparation of Simulated Body Fluid (SBF)

The preparation of SBF was carried out according to the method of Kokubo, T., et al., J. Biomed. Mater. Res., 1990. 24: p. 721-734.

The reagents shown in Table A were added, in order, to deionised water, to make 1 litre of SBF. All the reagents were dissolved in 700 ml of deionised water and warmed to a temperature of 37° C. The pH was measured and HCl was added to give a pH of 7.25 and the volume made up to 1000 ml with deionised water.

TABLE A
Reagents for the preparation of SBF

| Order | Reagents | Amount | |
|---|---|---|---|
| 1 | NaCl | 7.996 | g |
| 2 | NaHCO₃ | 0.350 | g |
| 3 | KCl | 0.224 | g |
| 4 | K₂HPO₄•3H₂O | 0.228 | g |
| 5 | MgCl₂•6H₂O | 0.305 | g |
| 6 | 1N HCL | 35 | ml |
| 7 | CaCl₂•2H₂O | 0.368 | g |
| 8 | Na₂SO4 | 0.071 | g |
| 9 | (CH₂OH)CNH₂ | 6.057 | g |

### Powder Assay to Determine Bioactivity:

Glass powder was added to 50 ml of Tris-Buffer solution or SBF and shaken at 37° C. At a series of time intervals, a sample was removed and the concentration of ionic species was determined using Inductively Coupled Plasma Emission Spectroscopy according to known methods (eg. Kokubo 1990).

In addition, the surface of the glass is monitored for the formation of an HCA layer by X-ray powder diffraction and Fourier Transform Infra Red Spectroscopy (FTIR). The appearance of hydroxycarbonated apatite peaks, characteristically at two theta values of 25.9, 32.0, 32.3, 33.2, 39.4 and 46.9 in an X-ray diffraction pattern is indicative of formation of a HCA layer. These values will be shifted to some extent due to carbonate substitution and Sr substitution in the lattice. The appearance of a P—O bend signal at a wavelength of 566 and 598 cm⁻¹ in an FTIR spectra is indicative of deposition of an HCA layer.

### Example 1

### Compositions of Strontium Containing Glasses

Table 1 below lists a number of melt-derived bioactive glass compositions, those of which that contain strontium are glasses of the present invention. Values of components are in mole percent.

TABLE 1

| | Application | SiO₂ | P₂O₅ | B₂O₃ | CaO | SrO | Na₂O | K₂O | ZnO | MgO | CaF₂ |
|---|---|---|---|---|---|---|---|---|---|---|---|
| 1 | Implant | 49.46 | 1.07 | 0 | 23.08 | 0 | 26.38 | | | | |
| | material/ | | | | | | | | | | |
| | Peridontal | | | | | | | | | | |
| | treatment | | | | | | | | | | |
| 2 | | 49.46 | 1.07 | 0 | 22.50 | 0.58 | 26.38 | | | | |
| 3 | | 49.46 | 1.07 | 0 | 20.77 | 2.31 | 26.38 | | | | |
| 4 | | 49.46 | 1.07 | 0 | 17.31 | 5.77 | 26.38 | | | | |
| 5 | | 49.46 | 1.07 | 0 | 11.54 | 11.54 | 26.38 | | | | |
| 6 | | 49.46 | 1.07 | 0 | 5.77 | 17.31 | 26.38 | | | | |
| 7 | | 49.46 | 1.07 | 0 | 0.00 | 23.08 | 26.38 | | | | |
| 8 | | 49.46 | 1.07 | 0 | 9.54 | 9.54 | 26.38 | | 2.0 | 2.0 | |
| 9 | | 49.46 | 1.07 | 0 | 9.54 | 9.54 | 13.19 | 13.19 | 2.0 | 2.0 | |
| 10 | | 47.46 | 1.07 | 2.0 | 9.54 | 9.54 | 13.19 | 13.19 | 2.0 | 2.0 | |
| 11 | | 49.46 | 1.07 | 0 | 9.54 | 9.54 | 6.60 | 13.19 | 2.0 | 2.0 | 6.60 |
| 12 | Bioactive | 49.46 | 1.07 | 0 | 27.27 | 3.00 | 6.6 | 6.60 | 3.00 | 3.00 | |
| | glass for | | | | | | | | | | |
| | Porous | | | | | | | | | | |
| | Sintered | | | | | | | | | | |
| | Scaffold | | | | | | | | | | |
| 13 | | 49.46 | 1.07 | 0 | 27.27 | 3.00 | 6.6 | 6.60 | 3.00 | 3.00 | |
| 14 | | 49.46 | 1.07 | 0 | 27.27 | 3.00 | 6.6 | 6.60 | 3.00 | 3.00 | |
| 15 | | 49.46 | 1.07 | 0 | 27.27 | 5.00 | 4.6 | 4.60 | 3.00 | 3.00 | |
| 16 | | 47.46 | 1.07 | 2.0 | 27.27 | 5.00 | 4.60 | 4.60 | 3.00 | 3.00 | |
| 17 | | 49.46 | 1.07 | 0 | 17.27 | 15.00 | 4.60 | 4.60 | 3.00 | 3.00 | |
| 18 | | 47.46 | 1.07 | 2.0 | 8.64 | 15.00 | 6.60 | 6.60 | 3.00 | 3.00 | 8.64 |
| 19 | Filler for | 49.46 | 1.07 | 0 | 21.43 | 21.43 | 6.6 | | | | |
| | Composites | | | | | | | | | | |
| 20 | | 49.46 | 1.07 | 0 | 21.43 | 21.43 | 6.6 | | | | |
| 21 | | 49.46 | 1.07 | 0 | 19.43 | 19.43 | 6.6 | | 2.00 | 2.00 | |
| 22 | | 49.46 | 1.07 | 0 | 19.43 | 19.43 | 3.3 | 3.3 | 2.00 | 2.00 | |
| 23 | Filler for | 49.46 | 1.07 | 0 | 9.72 | 19.43 | 3.3 | 3.3 | 2.00 | 2.00 | 9.72 |
| | Dental | | | | | | | | | | |
| | Tooth | | | | | | | | | | |
| | Filling | | | | | | | | | | |
| 24 | Glass for | 49.46 | 1.07 | 0 | 9.43 | 18.43 | 6.6 | | 3.00 | 10.00 | |
| | Polyacid | | | | | | | | | | |
| | Cement | | | | | | | | | | |
| 25 | | 49.46 | 1.07 | | 9.43 | 8.43 | 6.6 | | 3.00 | 20.00 | |
| 26 | | 51.46 | 1.07 | | 7.43 | 8.43 | 6.6 | | 3.00 | 20.00 | |
| 27 | | 53.53 | 0 | | 7.43 | 8.43 | 6.6 | | 3.00 | 20.00 | |
| 28 | Coating | 49.46 | 1.07 | | 29.02 | | 13.19 | | | 7.25 | |
| | (e.g. for | | | | | | | | | | |
| | Ti6Al4V) | | | | | | | | | | |
| 29 | | 49.46 | 1.07 | | 16.31 | 16.31 | 3.30 | 3.30 | 3.00 | 7.25 | |
| 30 | | 49.46 | 1.07 | | 13.01 | 13.01 | 3.30 | 3.30 | 3.00 | 13.85 | |
| 31 | | 49.46 | 1.07 | | 10.01 | 10.01 | 3.30 | 3.30 | 3.00 | 13.85 | 6.00 |
| 32 | | 49.46 | 1.07 | | 10.01 | 10.01 | 5.30 | 5.30 | 3.00 | 13.85 | |
| 33 | | 49.46 | 1.07 | | 8.51 | 8.51 | 6.60 | 6.60 | 3.00 | 16.25 | |
| 34 | | 49.46 | 1.07 | | 8.51 | 8.51 | 6.60 | 6.60 | 3.00 | 16.25 | |
| 35 | Bioactive | 49.46 | 1.07 | 0 | 0.00 | 20.08 | 26.38 | | 3.00 | | |
| | glass | | | | | | | | | | |
| | Toothpaste/ | | | | | | | | | | |
| | Deodorant | | | | | | | | | | |
| 36 | Bioactive | 49.46 | 1.07 | 0 | 0.00 | 16.08 | 26.38 | | 3.00 | | 4.00 |
| | glass | | | | | | | | | | |
| | Toothpaste | | | | | | | | | | |

As indicated in Table 1, certain bioactive glass composition are particularly suited to use in certain applications. For example, it has been found that glass compositions 12 to 18 and 28 to 34 as well as being of use for formation of implant material or in periodontal treatment or for use as a coating as indicated above, are also particularly useful for sintering and for drawing into fibres due to their large processing window.

### Example 2

### Bioactive Glass Powders and Monoliths

### Preparation of Glass No 5 as Listed in Table 1:

59.35 g of silica in the form of quartz, 3.04 g of phosphorus pentoxide, 23.08 g of calcium carbonate 34.07 g of strontium carbonate and 55.93 g of sodium carbonate are mixed together and placed in a platinum crucible and melted at 1390° C. for 1.5 hours then poured into demineralised water to produce a granular glass frit. The frit is dried the ground in a vibratory mill to produce a powder. The powder is sieved through a 45 micron mesh sieve. Of the sub 45 micron powder, 0.075 g was placed in 50 ml of simulated body fluid. The ability to form a calcium carbonated apatite (HCA) layer on its surface is a recognised test of a bioactive material. The glass was found to form an HCA layer on its surface by X-ray powder diffraction and Fourier Transform Infra Red Spectroscopy in less than six hours.

A corresponding synthetic method was carried out to prepared glasses 1 to 7 as set out in Table 1 and studies on these glasses demonstrated that the rate of formation of the carbonated apatite increased with increasing strontium substitution for calcium. The X-ray diffraction pattern of glasses 1 (no Sr) and 7 (with Sr) after immersion in SBF for 480 mins shown in FIG. 1 indicates that HCA formation is more pronounced with the strontium-containing glass.

The strontium-containing glass also precipitates calcium carbonate (peak marked ‘+’), once all the phosphate in the SBF has been used to form HCA.

In addition, the results of Tris-Buffer dissolution studies on glasses 1, 2, 3, 5 and 7 are shown in FIG. 2. Moreover, FIG. 6 shows a series of FTIR spectra of glass 7 after incubation in SBF for time periods between 0 and 480 minutes. The lowest trace represents unreacted glass and moving up FIG. 6, the traces represent glass reacted for 5, 15, 30, 60, 120, 240 and 480 minutes respectively. Over time the appearance of a P—O bend signal indicative of HCA layer formation is observed.

### Example 3

### Scaffold

### Preparation of Glass No 12 as Listed in Table 1:

59.35 g of silica in the form of quartz, 3.04 g of phosphorus pentoxide, 54.54 g of calcium carbonate 8.86 g of strontium carbonate and 13.99 g of sodium carbonate 18.24 g of potassium carbonate 4.88 g of zinc oxide and 2.42 g of magnesium oxide are mixed together and placed in a platinum crucible and melted at 1440° C. for 1.5 hours then poured into demineralised water to produce a granular glass frit. The frit is dried the ground in a vibratory mill to produce a powder. The powder was sieved through a 45 micron mesh sieve. The powder was then mixed with 50% by volume of approximately 200 micron suspension polymerised poly(methylmethacrylate) powder and pressed. The resulting pellet was fired by heating at 3° C. min⁻¹ to 700° C. with a 10 minute hold. The final material was amorphous when examined by X-ray diffraction and consisted of a porous interconnected solid. The pellet was found to form an HCA on its surface within 3 days when placed in simulated body fluid.

This is demonstrated by FIG. 7, in which a series of FTIR spectra of glass 12 after incubation in SBF for 0, 0.1, 0.3, 1, 5, 7 and 14 days is set out. Over time, the appearance of a P—O bend signal, indicative of HCA layer formation is observed.

### Example 4

### Bioactive Glass Coating with a TEC match to Ti6Al4V Alloy

### Preparation of Glass No 29 as Listed in Table 1:

59.35 g of silica in the form of quartz, 3.04 g of phosphorus pentoxide, 32.62 g of calcium carbonate 48.15 g of strontium carbonate and 6.96 g of sodium carbonate 9.12 g of potassium carbonate 4.88 g of zinc oxide and 5.84 g of magnesium oxide are mixed together and placed in a platinum crucible and melted at 1440° C. for 1.5 hours then poured into demineralised water to produce a granular glass frit. The frit is dried then ground in a vibratory mill to produce a powder. The powder is sieved through a 45 micron mesh sieve. A coating on Ti6Al4V is then produced by dispersing the glass powder in alcohol coating the suspension on to the metal and firing in an oxygen free environment at a heating rate of 3° C. min⁻¹ to 880° C. followed by a 15 minute hold followed by cooling back to room temperature. The coating was found to be crack free and well bonded to the metal and was found to form an HCA on its surface within 3 days when placed in simulated body fluid.

This is demonstrated by FIG. 7, in which a series of FTIR spectra of glass 29, after incubation in SBF for 1, 3, 7 and 14 days, is set out. Over time, the appearance of a P—O bend signal, indicative of HCA layer formation is observed.

In order to determine the TEC a small sample of frit was cast in the form of a 25 mm rod and the glass transition temperature, softening point and TEC measured using dilatometry. The values were found to be 591° C., 676° C. and 11×10⁻⁶K⁻¹.

### Calculation of Network Connectivity.

Network connectivity can be calculated according to the method set out in Hill, J. Mater. Sci. Letts., 15, 1122-1125 (1996), but with the assumption that the phosphorus is considered to exist as a separate orthophosphate phase and is not as part of the glass network.

### Example 5

### Cell Culture Results

Glass numbers 1, 2, 3, 5 and 7 as listed in Table 1 were prepared. In these glasses 0%, 2.5%, 10%, 50% or 100% of the calcium was substituted by strontium. This is set out in Table 2 below:

TABLE 2

| Glass composition | | | | | | |
|---|---|---|---|---|---|---|
| number (see Table 1) | % Sr | SiO₂ | P₂O₅ | CaO | SrO | Na₂O |
| 1 | 0 | 49.46 | 1.07 | 23.08 | 0 | 26.38 |
| 2 | 2.5 | 49.46 | 1.07 | 22.50 | 0.58 | 26.38 |
| 3 | 10 | 49.46 | 1.07 | 20.77 | 2.31 | 26.38 |
| 5 | 50 | 49.46 | 1.07 | 11.54 | 11.54 | 26.38 |
| 7 | 100 | 49.46 | 1.07 | 0.00 | 23.08 | 26.38 |

### Cell Culture Results

SAOS-2 cells (osteoblasts obtained from an osteogenic sarcoma cell line) were cultured in DMEM medium containing 10% FBS, 1% L-Glutamine (2 mM), 1% antibiotic/antimycotic and seeded (10,000 cells/cm²) on either the bioactive glass of the present invention containing 0%, 2.5%, 10%, 50% or 100% strontium or control cell culture plastic for the determination of alkaline phosphatase (ALP) activity, mineralisation and cell viability (MTS assay), Bioactive glass was incubated overnight in fully supplemented DMEM media at 37° C.-5% CO₂ prior to cell culture.

### Determination of ALP Activity

After 7 days in culture with the bioactive glass, ALP activity was determined as described in Ball et al, Biomaterials, 2001, 22(4): 337-347. ALP activity (mM) was calculated per mg of protein in the sample as determined by the DC protein assay (Bio-Rad, UK) over time. The osteoblast-like cells were observed to produce significantly more ALP when cultured on a bioactive glass comprising 2.5% and 50% strontium compared with no strontium. Increased ALP activity is associated with osteoblast differentiation into a mature mineralising phenotype.

### Mineralization of Osteoblasts on Composite Foam Scaffolds

To identify the active sites of mineralization Tetracycline labelling was applied as described in Holy et al, Biomed. Mater. Res., 2000, 51(3): 376-382. SAOS cells were cultured on the strontium containing bioactive glass (as described above) for 27 days. Tetracycline (1 μM) was then added to the medium for 24 hours prior to fixation and analysis using a fluorescent microscope. Increased mineralization was observed in bioactive glass comprising 2.5% and 50% strontium. This is in accordance with increased alkaline phosphatase activity observed in these bioactive glass compositions (2.5% and 50%).

### Cell Viability

The MTT viability assay (standard assay as described in Gerlier et al, J. Immunol. Meth. 94(1-2): 57-63, 1986, using reagent available from Sigma (cat. M5655-500MG): Thiazolyl Blue Tetrazolium Bromide) revealed that the bioactive glass comprising strontium significantly stimulated cell growth.

### Example 6

### Production of Sol Gel-Derived Glass

### Experimental Procedures

A glass according to the present invention can be prepared by sol gel techniques known in the art. The process set out in U.S. Pat. No. 5,074,916 was modified to form a glass according to the present invention and the modified process is set out below.

The glasses of the present invention may be prepared from an alkoxysilane, preferably tetraethylorthosilane (“TEOS”), for phosphate containing glasses an alkoxyphosphate, preferably triethylphosphate (“TEP”), strontium nitrate and optionally calcium nitrate, zinc nitrate and/or magnesium nitrate, using sol-gel preparation techniques. The following compounds were used for the processing of strontia-calcia-silicate gel-glasses: TEOS, Si(OC₂H₅)₄, 98% and strontium nitrate and calcium nitrate tetrahydrate, Ca(NO₃)₂.4H₂O, ACS reagent. Deionised (DI) water was obtained from an instant purifier with pH 5.5 and nitric acid was used as the catalyst.

2N HNO₃ was added to DI water and gently stirred for 5 min. TEOS was then added in small amounts over a 30-minute period. This mixture is maintained for one hour to ensure complete hydrolysis and the progression of condensation. The strontium nitrate and calcium nitrate was then added to this mixture and allowed to dissolve. Pouring and casting was achieved an hour later. The sol was prepared at room temperature and cast into teflon moulds for gelation.

Both aging and drying of wet gels were conducted in a programmable oven. Aging of the gels took place at 60° C. for 72 hours. The moulds were transferred into the oven after the gelation period and the oven was programmed to heat up to 60° C. at a heating rate of 5° C./min. The drying of the gels was carried out in the same jar by loosening the screw lids to allow gas evaporation and heating the gels with a three-stage schedule listed in Table 3 below.

TABLE 3
Drying schedule

| Stage | Temperature (° C.) | Duration (Hr) | Gradient (° C./min−1) |
|---|---|---|---|
| 1 | 60 | 20 | 0.1 |
| 2 | 90 | 24 | 0.1 |
| 3 | 130 | 40 | 0.1 |

For phosphate containing glasses, the molar ratio of water to TEOS plus TEP (i.e., H₂O/(TEOS+TEP), hereinafter the “R ratio”) should be maintained between three and ten (preferably eight), to obtain complete hydrolysis, reasonable gelation times (1-2 days), reasonable aging and drying times (2-4 days), and to prepare monoliths of the higher silica compositions. It is known that the range of R ratio facilitates preparation of coatings (at low R ratios), monoliths (at intermediate R ratios) and powders (at high R ratios).

The glass components (TEOS, nitric acid and water) are mixed and although TEOS and water are initially immiscible, the solution becomes clear after 10-20 minutes.

After 60 minutes, TEP is added to the stirring solution if P₂O₅ is to be incorporated. The strontium nitrate, and calcium nitrate, zinc nitrate and/or magnesium nitrate if included, are added after another 60 minutes of mixing. After this period ammonium fluoride may be added if fluorine is to be incorporated in the gel glass.

The solution is then stirred for an additional hour, following which it is retained in a quiescent state for 20 minutes. During this period the material coalesces into a sol, which is thereafter introduced into containers for casting. The containers are sealed with tape and placed into an oven for gelation and aging at 60° C. for 54 hours.

The samples are then removed from the aging chamber, placed in a glass container with a loose cover and the container introduced into a drying oven. Although exact adherence to this schedule is not critical for powdered forms, a drying schedule must be rigidly adhered to in order to produce monoliths. Appropriate adjustment of the drying schedule to accommodate monolith production is well within the purview of one skilled in the art.

The dried gel is placed in a quartz crucible for further calcination heat treatment. The calcination is carried out in a furnace through which is passed a slow flow of dry nitrogen gas. The nitrogen is used to avoid the formation and crystallization of HCA or mixed strontium/calcium carbonates in P₂O₅ free compositions during the heat treatment.

Exemplary sol-gel derived bioactive glass compositions, those of which that contain strontium are glasses according to the invention, are detailed in Table 4 below.

TABLE 4
Sol-gel glass compositions (Values in mole percent)

| Glass | Acronym | SiO₂ | SrO | CaO | ZnO | MgO | P₂O₅ |
|---|---|---|---|---|---|---|---|
| 37 | 70/30Sr | 70 | 30 | | | | |
| 38 | 70/25/5SrCa | 70 | 25 | 5 | | | |
| 39 | 70/20/5/5SrCaZn | 70 | 20 | 5 | 5 | | |
| 40 | 70/15/5/5/5 | 70 | 16 | 4 | 5 | 5 | |
| 41 | 80/15/5 | 80 | 15 | 5 | | | |
| 42 | 65/30/5SrP2O5 | 65 | 25 | | | | 5 |
| 43 | S70/30Ca* | 70 | | 30 | | | |
| 44 | S70//15Ca/15Sr | 70 | 15 | 15 | | | |
| 45 | S70/30Sr | 70 | 30 | | | | |

Glasses 43 and 44 as shown in Table 4 were tested for bioactivity using the SBF assay. The formation of a HCA layer was monitored by X-ray diffraction after 8 hours. The mixed Ca/Sr glass (glass 44) was shown to be more bioactive than glass 43, producing more apatite. By X-ray diffraction, a down-shifted, doublet diffraction peak at approximately 32 two theta being observed due to the formation of a mixed Ca/Sr apatite on the surface.

Dissolution studies were also carried out on glass 43. Results of the Tris-Buffer and SBF dissolution assays are shown in FIGS. 9 and 10. These assays demonstrate very rapid release kinetics and support the formation of a mixed Ca/Sr apatite on the surface of the glass, agreeing with the observed X-ray diffraction data.

### Example 7

### Production of a Single Layer Coating

Glasses 28 to 32 as shown in Table 1 above were prepared using the melt quench technique. The glasses, prepared to have a particle size <38 microns with a mean particle size of 5-6 microns, were coated on to a Ti6Al4V alloy sheet (to act as a model for, for example, a Ti6Al4V hip implant) by mixing the glass with chloroform containing 1% poly(methylmethacrylate) of molecular weight 50,000 to 100,000 in a weight ratio of 1:10. The alloy sheet (or the femoral stem of the prostheses) is immersed in the chloroform glass suspension, drawn slowly out, and the chloroform evaporated off. The sheet (or prosthesis) is then heated at 2 to 60° C. min⁻¹ to 750° C., held for 30 mins, fired under vacuum before cooling to room temperature. The coated sheet has a glossy bioactive coating over the immersed area of between 50 and 300 microns thick. When placed in simulated body fluid the coating is observed to deposit a hydroxycarbonated apatite layer in under 3 days. This technique can be applied to other alloys and ceramics such as Al₂O₃ and Zirconia.

### Example 8

### Production of a Bilayer Coating for Ti6Al4V

Optimum bioactivity is required to promote osseointegration. However it is also desirable that the Ti6Al4V remains coated after long time periods in the body. For this reason it is desirable to have a much less reactive base glass layer and a more reactive top coat layer. In this context, less reactive glass has lower bioactivity and higher chemical stability, and more reactive glass has higher bioactivity and lower chemical stability. Such coatings can be fabricated by a two step process as summarized below.

A glass taken from Table 5 below (not a bioactive glass of the present invention), having a particle size <38 microns with a mean particle size of 5-6 microns, is coated on to a Ti6Al4V alloy hip implant by mixing the glass with chloroform containing 1% polymethylmethacrylate of molecular weight 50,000 to 100,000 in a weight ratio of 1:10. The femoral stem of the prostheses is immersed in the chloroform glass suspension drawn slowly out and the chloroform evaporated off.

TABLE 5
(Compositions in molar percent)

| Glass | SiO₂ | P₂O₅ | CaO | Na₂O | K₂O | MgO |
|---|---|---|---|---|---|---|
| 1 | 61.34 | 2.55 | 13.55 | 10.01 | 1.79 | 10.56 |
| 2 | 68.40 | 2.56 | 10.93 | 4.78 | 6.78 | 6.57 |
| 3 | 67.40 | 2.56 | 11.93 | 4.78 | 6.78 | 6.57 |

The process is repeated with a second glass taken from the Table 1 above. The prosthesis is then heated at 2 to 60° C. min⁻¹ to 750° C., held for 30 mins and fired under vacuum before cooling to room temperature.

The coated prosthesis has a glossy bioactive coating over the immersed area of between 50 and 300 microns thick.

### Example 9

### Production of Bilayer Coatings for Chrome Cobalt Alloys

Bilayer coatings on chrome cobalt are particularly desirable since there can be significant dissolution of the oxides of cobalt nickel and chromium from the protective oxide layer into the glass which could be released from the glass. For this reason a chemically stable base coating glass composition is preferred.

A glass of the composition taken from Table 6 (not a bioactive glass of the present invention) having a particle size <38 microns with a mean particle size of 5-6 microns is coated on to a Chrome Cobalt alloy hip implant by mixing the glass with chloroform containing 1% polymethylmethacrylate of molecular weight 50,000 to 100,000 in a weight ratio of 1:10. The femoral stem of the prosthesis is immersed in the chloroform glass suspension drawn slowly out and the chloroform evaporated off.

TABLE 6
(Compositions in molar percent)

| Glass | SiO₂ | CaO | Na₂O | K₂O | ZnO | MgO |
|---|---|---|---|---|---|---|
| 1 | 61.10 | 22.72 | 12.17 | 4.00 | 0.00 | 0.00 |
| 2 | 66.67 | 6.28 | 7.27 | 10.62 | 4.47 | 4.70 |
| 3 | 68.54 | 14.72 | 9.11 | 7.63 | 0.00 | 0.00 |
| 4 | 66.67 | 15.56 | 9.29 | 7.24 | 0.23 | 0.00 |

The process is then repeated with a bioactive glass having a composition taken from Table 7.

TABLE 7
(Compositions in molar percent)

| Glass | SiO₂ | P₂O₅ | B₂O₃ | CaO | SrO | Na₂O | K₂O | ZnO | MgO | CaF₂ |
|---|---|---|---|---|---|---|---|---|---|---|
| 46 | 49.09 | 8.42 | 0.00 | 4.21 | 4.21 | 8.65 | 8.72 | 8.34 | 8.35 | 0.00 |
| 47 | 45.00 | 3.00 | 0.00 | 10.00 | 10.00 | 10.0 | 8.00 | 4.00 | 10.00 | 0.00 |
| 48 | 50.00 | 3.00 | 0.00 | 7.50 | 7.50 | 10.0 | 8.00 | 4.00 | 10.00 | 0.00 |
| 49 | 49.00 | 3.00 | 0.00 | 7.50 | 7.50 | 10.0 | 8.00 | 4.00 | 10.00 | 0.00 |
| 50 | 46.00 | 3.00 | 0.00 | 11.50 | 11.50 | 8.00 | 7.00 | 3.00 | 10.00 | 0.00 |
| 51 | 45.00 | 3.00 | 0.00 | 15.00 | 5.00 | 8.00 | 7.00 | 3.00 | 10.00 | 4.00 |
| 52 | 45.00 | 2.00 | 2.00 | 15.00 | 9.00 | 8.00 | 7.00 | 2.00 | 9.00 | 0.00 |

1-42. (canceled)

43. An aluminium-free bioactive glass comprising Sr and SiO₂, wherein the Sr is provided as SrO and the molar percentage of SrO is 0.2% to 45%.

44. The bioactive glass of claim 43 further comprising a source of one or more of Na, K, Ca, P₂O₅, Mg, Zn, B₂O₃, F, or Ag.

45. The bioactive glass of claim 44 wherein the F is provided as one or more of CaF₂, SrF₂, MgF₂, NaF, or KF and the combined molar percentage of CaF₂, SrF₂, MgF₂, NaF, and KF is 0% to 50%.

46. The bioactive glass of claim 44 which comprises any one or more of:
a source of Na ions and/or a source of K ions at a combined molar percentage of 0% to 30%;
CaO at a molar percentage of 0% to 50%;
P₂O₅ at a molar percentage of 0% to 14%;
MgO at a molar percentage of 0% to 40%;
ZnO at a molar percentage of 0% to 10%; and
B₂O₃ at a molar percentage of 0% to 15%.

47. The bioactive glass of claim 46 wherein the glass comprises:
approximately 46% to 50% SiO₂;
approximately 0.5% to 1.5% P₂O₅;
approximately 0% to 2% B₂O₃;
approximately 0% to 23% CaO;
approximately 0.5% to 24% SrO;
approximately 6% to 27% Na₂O;
approximately 0% to 13% K₂O;
approximately 0% to 2% ZnO;
approximately 0% to 2% MgO; and
approximately 0% to 7% CaF₂.

48. The bioactive glass of claim 46 wherein the glass comprises:
approximately 49% to 50% SiO₂;
approximately 0.5% to 1.5% P₂O₅;
approximately 8% to 30% CaO;
approximately 8% to 17% SrO;
approximately 3% to 7% Na₂O;
approximately 3% to 7% K₂O;
approximately 3% ZnO;
approximately 7% to 16% MgO; and
approximately 0% to 6% CaF₂.

49. The bioactive glass of claim 43 wherein the bioactive glass is a melt-derived bioactive glass and wherein the molar percentage of SiO₂ is 30% to 60%.

50. The bioactive glass of claim 49 wherein the combined molar percentage of SiO₂, P₂O₅, and B₂O₃ does not exceed 60%.

51. The bioactive glass of claim 49 wherein the combined molar percentage of SrO, CaO, MgO, Na₂O₁ and K₂O is 40% to 60%.

52. The bioactive glass of claim 43 wherein the bioactive glass is a sol gel-derived bioactive glass wherein the molar percentage of SiO₂ is 50% to 95%.

53. The bioactive glass of claim 43 wherein the bioactive glass is in particulate form, is provided as fibres, or comprises a solid.

54. The bioactive glass of claim 53 wherein the solid is a disk or monolith.

55. A process for the production of a bioactive glass of claim 43 comprising admixing Sr and SiO₂ and optionally one or more of Na, K, Ca, P₂O₅, Mg, Zn, B₂O₃, F, or Ag.

56. A composition comprising a bioactive glass of claim 43.

57. The composition of claim 56 wherein the composition is bone cement, a dental composite, a degradable polymer, a bioactive porous scaffold, a toothpaste, a deodorant, a bone substitute, a powder, a bioactive glass filled acrylic, a bioactive glass filled polylactide, a bioactive glass filled Bis GMA or dental composite, a bioactive glass granule, a sintered bioactive glass, or an implant coating.

58. The composition of claim 57 wherein the composition is an implant coating and the coating comprises two or more layers, wherein at least one layer comprises the bioactive glass.

59. An implant coated with the coating of claim 56.

60. The implant of claim 58 which is a joint prosthesis.

61. A method of preventing and/or treating damage to a tissue comprising administering a bioactive glass of claim 43 to a patient in need thereof.

62. The method of claim 61 wherein the tissue comprises bone or dental tissue.

63. The method of claim 61 wherein the administration of the bioactive glass is parenteral, oral, or topical.

64. The method of claim 61 wherein the tissue damage is a bone fracture, a dental carie, a periodontal disease, a hypersensitive tooth, or a demineralised tooth.

65. A method of increasing the rate of hydroxycarbonated apatite deposition comprising administering a bioactive glass of claim 43 to a patient in need thereof.
